(19) United States
(12) Patent Application Publication
(10) Pub. No.: US 2015/0247161 A1
(43) Pub. Date: Sep. 3, 2015

(54) METHOD OF PRODUCING STRESS TOLERANT PLANTS OVER-EXPRESSING OsAlba1

(71) Applicant: National Institute of Plant Genome Research, New Delhi (IN)

(72) Inventors: Niranjan Chakraborty, New Delhi (IN); Subhra Chakraborty, New Delhi (IN); Jitendra Kumar Verma, New Delhi (IN); Saurabh Gayali, New Delhi (IN); Suchismita Dass, New Delhi (IN); Amit Kumar, New Delhi (IN); Shaista Parveen, New Delhi (IN)

(21) Appl. No.: 14/581,002

(22) Filed: Dec. 23, 2014

(30) Foreign Application Priority Data
Dec. 24, 2013 (IN) 3759/DEL/2013

(51) Int. Cl. C12N 15/82; C07K 14/415

(52) U.S. Cl. CPC C12N 15/8273; C07K 14/415; C12N 15/8271

(57) ABSTRACT

In the present disclosure there is provided transgenic plants that are resistant to a host of abiotic stress factors. In particular, the said transgenic plants over-express the translated product of OsAlba1 gene. Also provided in the present disclosure are cDNA fragments, recombinant DNA constructs, vectors and host cells useful in generating transgenic plants that are resistant to abiotic stress susceptibility.

### CLAIM OF PRIORITY

This application claims the benefit of priority of India Patent Application No. 3759/DEL/2013, filed on Dec. 24, 2013, the benefit of priority of which is claimed hereby, and which is incorporated by reference herein in its entirety.

### FIELD OF INVENTION

The present disclosure relates to a method of producing transgenic plants over-expressing OsAlba1 gene. The transgenic plants have enhanced stress tolerance. The disclosure also provides cDNA, recombinant DNA constructs, recombinant DNA vectors, and recombinant host cells comprising the OsAlba1 cDNA.

### BACKGROUND OF THE INVENTION

Availability of water is the most crucial factor that limits the productivity potential of higher plants and is a serious threat to sustainable crop production. In order to improve the agricultural productivity under water-deficit condition or dehydration, it is required to first understand the biochemical and molecular mechanisms by which plants tolerate such stress. Extensive investigations at the molecular level of plant response to environmental stresses have led to the cloning of various stress inducible genes (Ingram et al., Annu. Rev. Plant Physiol. Plant Mol. Biol., 1996, 47, 377-403; Bray, Trends Plant Sci., 1997, 2, 48-54). Nevertheless, the significance of these genes in stress tolerance is incomplete without the knowledge of their function. Most studies on stress adaptation have mainly focused on the changes in gene expression, while far less information is available on their protein counterparts. Rice is the most important food cereal, with nearly half of the world's population relying on its successful harvests. While rice is the first crop plant to have its complete genome sequenced (Goff et al., Science, 2002, 296, 92-100; Yu et al., Science, 2002, 296, 79-92), the functions of most of the predicted genes are unknown. Therefore, characterization of these unknown genes is one of the major challenges of the rice functional genomics.

The nucleus is a highly organized organelle that contains specific functional domains essential for the regulated expression of proteins in eukaryotes. The fact that nearly one-fourth of total cellular proteins are localized in the nucleus implies a variety of function in this compartment (Kumar et al., Genes Dev. 2002, 16, 707-719). Though it is an attractive target for the study of cellular homeostasis and determination of genomic response to stress, the plant nuclear proteomics is still in its infancy, particularly with respect to stress tolerance. Of the few plant nuclear matrix proteins characterized, no obvious homology was found with known nuclear proteins in yeast and mammals (Gindullis et al., Plant Cell, 1999, 11, 1117-1128; Gindullis et al., Plant Cell, 1999, 11, 1755-1768). Interestingly, Alba (acetylation lowers binding affinity) proteins, first identified as DNA-binding proteins in archaea, and then in association with nuclear RNase P/MRP in yeast and mammalian cells, are part of a superfamily that spans through three domains of life (Mani et al., 2011). The members of Alba family proteins have been reported to play important role in transcriptional regulation by maintaining chromatin architecture (Aravind et al., Genome Biol. 4, 2003, R64). The dimeric protein, formerly named Sso10b, is conserved in most sequenced archaeal genomes (Forterre et al., Mol. Microbiol., 1999, 32, 669-670) and binds double-stranded DNA tightly, but without apparent sequence specificity (Grote et al., Biochim. Biophys. Acta, 1986, 873, 405-413; Xue et al., J. Bacteriol., 2000, 182, 3929-3933; Bell et al., Science, 2002, 296, 148-151). As evident from its name, Alba is controlled by acetylation and deacetylation, where acetylation at specific N-terminal lysine residue lowers its binding affinity toward double-stranded DNA (Bell et al., Science, 2002, 296, 148-151; Zhao et al., J. Biol. Chem., 2003, 278, 26071-26077). In Sulfolobus solfataricus, Alba was found to be reversibly acetylated at Lys-16 by a homolog of Pat (protein acetyltransferase) and deacetylated by Sir2 protein, a NAD⁺-dependent deacetylase belonging to sirtuin family (Bell et al., Science, 2002, 296, 148-151; Starai et al., J. Mol. Biol., 2004, 340, 1005-1012; Marsh et al., J. Biol. Chem., 2005, 280, 21122-21128). The best-studied eukaryotic members of the family are the human and yeast proteins Rpp20/Pop7 and Rpp25/Pop6 (Perederina et al., RNA, 2010, 13, 1648-1655 2007; Hands-Taylor et al., Nucleic Acids Res., 2010, 38, 4052-4066). A recent study suggested that they are only transiently associated with RNase MRP in vivo and are not present in the catalytically active complex (Welting et al., RNA, 2006, 12, 1373-1382). By and large, however, their function remains far from understood.

PCT/IB2002/005253 discloses over-expression of MnSOD in crop plants for improved resistance against environmental stress conditions.

PCT/CN2012/074106 discloses over-expression of rice OXHS4 gene for improved resistance against drought tolerance.

PCT/EP2011/069367 discloses inactivation of MADS box gene function for improved resistance against biotic and/or abiotic stress.

### SUMMARY OF INVENTION

An aspect of the present disclosure provides a recombinant DNA construct comprising of a promoter operably linked to a gene of interest, wherein said gene of interest encodes a polypeptide having amino acid sequence as set forth in SEQ ID NO: 1.

An aspect of the present disclosure provides a recombinant DNA vector comprising a recombinant DNA construct comprising of a promoter operably linked to a gene of interest, wherein said gene of interest encodes a polypeptide having amino acid sequence as set forth in SEQ ID NO: 1.

An aspect of the present disclosure provides a recombinant host cell comprising a recombinant DNA construct comprising of a promoter operably linked to a gene of interest, wherein said gene of interest encodes a polypeptide having amino acid sequence as set forth in SEQ ID NO: 1.

An aspect of the present disclosure provides a recombinant host cell comprising a recombinant DNA vector, said recombinant DNA vector comprising a recombinant DNA construct comprising of a promoter operably linked to a gene of interest, wherein said gene of interest encodes a polypeptide having amino acid sequence as set forth in SEQ ID NO: 1.

An aspect of the present disclosure provides a cDNA fragment encoding a polypeptide having amino acid sequence as set forth in SEQ ID NO: 1, wherein cells expressing said polypeptide exhibit enhanced abiotic stress tolerance selected from the group consisting of ABA induced stress, salt stress, thermal stress, dehydration stress, oxidative stress, and combinations thereof.

An aspect of the present disclosure provides a method of producing a transgenic plant with enhanced tolerance to abiotic stress, said method comprising: (a) transforming plant cells with recombinant DNA construct comprising of a promoter operably linked to a gene of interest, wherein said gene of interest encodes a polypeptide having amino acid sequence as set forth in SEQ ID NO: 1; or recombinant host cells comprising a recombinant DNA vector comprising, said recombinant DNA vector comprising of a recombinant DNA construct comprising of a promoter operably linked to a gene of interest, wherein said gene of interest encodes a polypeptide having amino acid sequence as set forth in SEQ ID NO: 1; (b) selecting a transgenic cell, and (c) developing a transgenic plant from said transformed cell, wherein said transgenic plant exhibits enhanced tolerance to abiotic stress.

An aspect of the present disclosure provides transgenic plant or parts thereof, including seeds, wherein said transgenic plant or parts thereof, including seeds, encodes at the nuclear genome level a polynucleotide fragment comprising of a cDNA fragment encoding a polypeptide having amino acid sequence as set forth in SEQ ID NO: 1, wherein expression of said polypeptide confers abiotic stress resistance to said transgenic plant or parts thereof, including seeds.

In an aspect of the present disclosure, there is provided a use of a cDNA fragment in generating transgenic plants that are resistant to abiotic stress, wherein said cDNA fragment encodes a polypeptide having amino acid sequence as set forth in SEQ ID NO: 1.

### BRIEF DESCRIPTION OF ACCOMPANYING DRAWINGS

The following drawings form part of the present specification and are included to further illustrate embodiments of the present invention. The invention may be better understood by reference to the drawings in combination with the detailed description of the specific embodiments presented herein.

FIG. 1 shows the graphical representation of OsAlba1 transcript levels in various plant parts, in accordance with an embodiment of the present disclosure.

FIG. 2 shows OsAlba1 protein levels in various plant parts, in accordance with an embodiment of the present disclosure.

FIG. 3 shows the sub-cellular localization of OsAlba1, in accordance with an embodiment of the present disclosure.

FIG. 4 shows the results from OsAlba1 yeast complementation studies, in accordance with an embodiment of the present disclosure.

FIG. 5A shows the graphical representation of effect of dehydration on OsAlba1 transcript levels, in accordance with an embodiment of the present disclosure.

FIG. 5B shows the graphical representation of effect of dehydration on OsAlba1 protein levels, in accordance with an embodiment of the present disclosure.

FIG. 5C shows the graphical representation of effect of salt stress on OsAlba1 transcript levels, in accordance with an embodiment of the present disclosure.

FIG. 5D shows the graphical representation of effect of thermal stress on OsAlba1 transcript levels, in accordance with an embodiment of the present disclosure.

FIG. 5E shows the graphical representation of effect of ABA stress on OsAlba1 transcript levels, in accordance with an embodiment of the present disclosure.

FIG. 6 shows the structural organization of OsAlba1 gene, in accordance with an embodiment of the present disclosure.

FIG. 7 shows the representation of the pGA3426-OsAlba1 construct used for Agrobacterium mediated rice plant transformation, in accordance with an embodiment of the present disclosure.

### DETAILED DESCRIPTION OF THE INVENTION

Those skilled in the art will be aware that the invention described herein is subject to variations and modifications other than those specifically described. It is to be understood that the invention described herein includes all such variations and modifications. The invention also includes all such steps, features, compositions and compounds referred to or indicated in this specification, individually or collectively, and any and all combinations of any or more of such steps or features.

### DEFINITIONS

For convenience, before further description of the present invention, certain terms employed in the specification, example and appended claims are collected here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps. The term “including” is used to mean “including but not limited to”. “Including” and “including but not limited to” are used interchangeably.

“Primers” are synthesized nucleic acids that anneal to a complementary target DNA strand by hybridization to form a hybrid between the primer and the target DNA strand, and then extended along the target DNA strand by polymerase activity, e.g., a DNA polymerase. Primer pairs described in the present invention refer to their use for amplification of a target nucleic acid sequence, e.g., by polymerase chain reaction or other conventional nucleic-acid amplification methods.

SEQ ID NO: 1 shows the amino acid sequence of OsAlba1 protein.
MAVEEITEGVRNLAVEGEPAAAAAAAGGGGEGAQRRAAGSSSNRIQVSNT
KKPLFFYVNLAKRYMQQHGDVELSALGMAIATVVTVAEILKNNGFAVEKK
IRTSTVEINDESRVRPLQKAKIEIVLEKSEKFDELMAAAAEEREAAEAEEQA
SEQ ID NO: 2 shows the cDNA sequence encoding OsAlba1 protein.
ATGGCGGTGGAGGAGATCACCGAGGGGGTGAGGAACCTGGCCGTGGAG
GGGGAGCCCGCGGCGGCGGCGGCGGCGGCGGGAGGTGGTGGTGAGGGG
GCGCAGAGGAGGGCGGCCGGGAGCAGCAGCAACCGCATCCAGGTGTCC
AACACCAAGAAGCCACTCTTCTTCTATGTCAACCTCGCCAAGAGGTACA
TGCAGCAGCACGGCGATGTCGAGCTCTCCGCGCTCGGGATGGCCATTGC
AACAGTTGTAACTGTTGCGGAGATTCTTAAGAATAACG
GGTTTGCTGTTGAAAAGAAGATTAGAACATCTACGGTGGAAATAAACGA
TGAATCGAGAGTTCGCCCGCTCCAAAAGGCTAAGATTGAGATAGTGTTA
GAAAAGAGCGAGAAATTTGATGAGCTGATGGCTGCCGCAGCGGAAGAG
AGGGAAGCTGCGGAAGCTGAGGAGCAGGCCTGA
SEQ ID NO: 3 shows the forward primer sequence used to clone OsAlba1
gene.
CGCGGATCCATGGCGGTGGAGGAGATCA
SEQ ID NO: 4 shows the reverse primer sequence used to clone OsAlba1
gene.
ATAAGAATGCGGCCGCTCAGGCCTGCTCCTCA
SEQ ID NO: 5 shows the peptide sequence used to generate antibodies
against OsAlba1 protein.
RTSTVEINDESRVRP

In an embodiment of the present disclosure, there is provided a recombinant DNA construct comprising of a promoter operably linked to a gene of interest, wherein said gene of interest encodes a polypeptide having amino acid sequence as set forth in SEQ ID NO: 1.

In an embodiment of the present disclosure, there is provided a recombinant DNA construct as described herein, wherein said gene of interest polynucleotide sequence is as set forth in SEQ ID NO: 2.

In a preferred embodiment of the present disclosure, there is provided a recombinant DNA construct as described herein, wherein said gene of interest is OsAlba1.

In an embodiment of the present disclosure, there is provided a recombinant DNA construct as described herein, wherein said promoter is selected from the group consisting of pUbi, Cam V 35S, Act-1, Adh-1, and opine promoters.

In a preferred embodiment of the present disclosure, there is provided a recombinant DNA construct as described herein, wherein said promoter is pUbi.

In an embodiment of the present disclosure, there is provided a recombinant DNA vector comprising a recombinant DNA construct, said recombinant DNA construct comprising of a promoter operably linked to a gene of interest, wherein said gene of interest encodes a polypeptide having amino acid sequence as set forth in SEQ ID NO: 1.

In an embodiment of the present disclosure, there is provided a recombinant DNA vector as described herein, wherein said gene of interest polynucleotide sequence is as set forth in SEQ ID NO: 2.

In a preferred embodiment of the present disclosure, there is provided a recombinant DNA vector as described herein, wherein said gene of interest is OsAlba1.

In an embodiment of the present disclosure, there is provided a recombinant host cell comprising of a recombinant DNA construct, said recombinant DNA construct comprising of a promoter operably linked to a gene of interest, wherein said gene of interest encodes a polypeptide having amino acid sequence as set forth in SEQ ID NO: 1.

In an embodiment of the present disclosure, there is provided a recombinant host cell as described herein, wherein said gene of interest polynucleotide sequence is as set forth in SEQ ID NO: 2.

In a preferred embodiment of the present disclosure, there is provided a recombinant host cell as described herein, wherein said gene of interest is OsAlba1.

In an embodiment of the present disclosure, there is provided a recombinant host cell as described herein, wherein said recombinant host cell is of bacterial or fungal or of plant origin.

In an embodiment of the present disclosure, there is provided a recombinant host cell as described herein, wherein said recombinant host cell is of bacterial origin.

In an embodiment of the present disclosure, there is provided a recombinant host cell as described herein, wherein said recombinant host cell is of fungal origin.

In an embodiment of the present disclosure, there is provided a recombinant host cell as described herein, wherein said recombinant host cell is of plant origin.

In an embodiment of the present disclosure, there is provided a recombinant host cell comprising of a recombinant DNA vector, said recombinant DNA vector comprising of a recombinant DNA construct, said recombinant DNA construct comprising of a promoter operably linked to a gene of interest, wherein said gene of interest encodes a polypeptide having amino acid sequence as set forth in SEQ ID NO: 1.

In an embodiment of the present disclosure, there is provided a recombinant host cell as described herein, wherein said gene of interest has polynucleotide sequence as set forth in SEQ ID NO: 2.

In a preferred embodiment of the present disclosure, there is provided a recombinant host cell as described herein, wherein said gene of interest is OsAlba1.

In an embodiment of the present disclosure, there is provided a cDNA fragment encoding a polypeptide having amino acid sequence as set forth in SEQ ID NO: 1, wherein cells expressing said polypeptide exhibit enhanced abiotic stress tolerance selected from the group consisting of ABA induced stress, salt stress, thermal stress, dehydration stress, oxidative stress, and combinations thereof.

In an embodiment of the present disclosure, there is provided a cDNA fragment as described herein, wherein said cDNA fragment has polynucleotide sequence as set forth in SEQ ID NO: 2.

In an embodiment of the present disclosure, there is provided a method of producing a transgenic plant with enhanced tolerance to abiotic stress, said method comprising of (a) transforming plant cells with recombinant DNA construct comprising of a promoter operably linked to a gene of interest, wherein said gene of interest encodes a polypeptide having amino acid sequence as set forth in SEQ ID NO: 1, or recombinant host cells comprising of a recombinant DNA vector, said recombinant DNA vector comprising of a recombinant DNA construct, said recombinant DNA construct comprising of a promoter operably linked to a gene of interest, wherein said gene of interest encodes a polypeptide having amino acid sequence as set forth in SEQ ID NO: 1, (b) selecting a transgenic plant cell, and (c) developing a transgenic plant from said transformed cell, wherein said transgenic plant exhibits enhanced tolerance to stress.

In an embodiment of the present disclosure, there is provided a method of producing a transgenic plant with enhanced tolerance to abiotic stress as described herein, wherein said gene of interest polynucleotide sequence is as set forth in SEQ ID NO: 2.

In an embodiment of the present disclosure, there is provided a method of producing a transgenic plant with enhanced tolerance to abiotic stress as described herein, wherein abiotic stress is selected from the group consisting of ABA induced stress, salt stress, thermal stress, dehydration stress, oxidative stress, and combinations thereof.

In an embodiment of the present disclosure, there is provided a method of producing a transgenic plant with enhanced tolerance to abiotic stress as described herein, wherein transformation method is selected from the group consisting of Agrobacterium mediated transformation method, particle gun bombardment method, in-planta transformation method, liposome mediated transformation method, protoplast transformation method, microinjection, and macroinjection.

In a preferred embodiment of the present disclosure, there is provided a method of producing a transgenic plant with enhanced tolerance to abiotic stress as described herein, wherein transformation method is Agrobacterium mediated transformation method.

In an embodiment of the present disclosure, there is provided a method of producing a transgenic plant with enhanced tolerance to abiotic stress as described herein, wherein said transgenic plant is a monocot.

In an embodiment of the present disclosure, there is provided a method of producing a transgenic plant with enhanced tolerance to abiotic stress wherein said transgenic plant is a monocot as described herein, and wherein said monocot is selected from the group consisting of wheat, rice, banana, barley, millet, and rye.

In a preferred embodiment of the present disclosure, there is provided a method of producing a transgenic plant with enhanced tolerance to abiotic stress wherein said transgenic plant is a monocot as described herein, and wherein said monocot is rice.

In an embodiment of the present disclosure, there is provided a method of producing a transgenic plant with enhanced tolerance to abiotic stress as described herein, wherein said transgenic plant is a dicot.

In an embodiment of the present disclosure, there is provided a method of producing a transgenic plant with enhanced tolerance to abiotic stress wherein said transgenic plant is a dicot as described herein, wherein said dicot is selected from the group consisting of beans, peas, potato, eggplant, peppers, squash, melons, coffee, citrus, broccoli, turnips, yams, and apples.

In an embodiment of the present disclosure, there is provided a transgenic plant or parts thereof, including seeds with enhanced tolerance to abiotic stress, which encodes in its nuclear genome a cDNA fragment encoding a polypeptide having amino acid sequence as set forth in SEQ ID NO: 1, and wherein said cDNA fragment is operably linked to a promoter.

In an embodiment of the present disclosure, there is provided a transgenic plant or parts thereof, including seeds as described herein, wherein said nuclear encoded cDNA fragment has polynucleotide sequence as set forth in SEQ ID NO: 2.

In an embodiment of the present disclosure, there is provided a transgenic plant or parts thereof, including seeds as described herein, wherein said transgenic plant or parts thereof, including seeds is a monocot.

In an embodiment of the present disclosure, there is provided a transgenic plant or parts thereof, including seeds wherein said transgenic plant or parts thereof including seeds is a monocot as described herein, wherein said monocot is selected from the group consisting of wheat, rice, banana, barley, millet, and rye.

In a preferred embodiment of the present disclosure, there is provided a transgenic plant or parts thereof, including seeds wherein said transgenic plant or parts thereof including seeds is a monocot as described herein, wherein said monocot is rice.

In an embodiment of the present disclosure, there is provided a transgenic plant or parts thereof, including seeds as described herein, wherein said transgenic plant or parts thereof, including seeds is a dicot.

In an embodiment of the present disclosure, there is provided a transgenic plant or parts thereof, including seeds wherein said transgenic plant or parts thereof including seeds is a dicot as described herein, wherein said dicot is selected from the group consisting of beans, peas, potato, eggplant, peppers, squash, melons, coffee, citrus, broccoli, turnips, yams, and apples,

In an embodiment of the present disclosure, there is provided a transgenic plant or parts thereof, including seeds as described herein, wherein said transgenic plant or parts thereof, including seeds are resistant to ABA induced stress, salt stress, dehydration stress, thermal stress, oxidative stress, or combinations thereof.

In an embodiment of the present disclosure, there is provided a use of a cDNA fragment encoding a polypeptide having amino acid sequence as set forth in SEQ ID NO: 1 to develop transgenic plants with enhanced tolerance to abiotic stress.

In an embodiment of the present disclosure, over-expression of a polypeptide having amino acid sequence as set forth in SEQ ID NO: 1 results in enhanced tolerance to oxidative stress.

In an embodiment of the present disclosure, over-expression of a polypeptide encoded by a polynucleotide sequence as set forth in SEQ ID NO: 2 results in enhanced tolerance to oxidative stress.

In an embodiment of the present disclosure, over-expression of a polypeptide having amino acid sequence as set forth in SEQ ID NO: 1 results in enhanced tolerance to salt stress.

In an embodiment of the present disclosure, over-expression of a polypeptide encoded by a polynucleotide sequence as set forth in SEQ ID NO: 2 results in enhanced tolerance to salt stress.

In an embodiment of the present disclosure, over-expression of a polypeptide having amino acid sequence as set forth in SEQ ID NO: 1 results in enhanced tolerance to thermal stress.

In an embodiment of the present disclosure, over-expression of a polypeptide encoded by a polynucleotide sequence as set forth in SEQ ID NO: 2 results in enhanced tolerance to thermal stress.

In an embodiment of the present disclosure, over-expression of a polypeptide having amino acid sequence as set forth in SEQ ID NO: 1 results in enhanced tolerance to osmotic stress.

In an embodiment of the present disclosure, over-expression of a polypeptide encoded by a polynucleotide sequence as set forth in SEQ ID NO: 2 results in enhanced tolerance to osmotic stress.

In an embodiment of the present disclosure, expression of the polypeptide having amino acid sequence as set forth in SEQ ID NO: 1 is induced under oxidative stress.

In an embodiment of the present disclosure, expression of a polypeptide encoded by a polynucleotide sequence as set forth in SEQ ID NO: 2 is induced under oxidative stress.

In an embodiment of the present disclosure, expression of the polypeptide having amino acid sequence as set forth in SEQ ID NO: 1 is induced under thermal stress.

In an embodiment of the present disclosure, expression of a polypeptide encoded by a polynucleotide sequence as set forth in SEQ ID NO: 2 is induced under thermal stress.

In an embodiment of the present disclosure, expression of the polypeptide having amino acid sequence as set forth in SEQ ID NO: 1 is induced under osmotic stress.

In an embodiment of the present disclosure, expression of a polypeptide encoded by a polynucleotide sequence as set forth in SEQ ID NO: 2 is induced under osmotic stress.

In an embodiment of the present disclosure, expression of the polypeptide having amino acid sequence as set forth in SEQ ID NO: 1 is induced under salt stress.

In an embodiment of the present disclosure, expression of a polypeptide encoded by a polynucleotide sequence as set forth in SEQ ID NO: 2 is induced under salt stress.

In an embodiment of the present disclosure, there is provided a transgenic plant or parts thereof or seeds or progeny that express a polypeptide having amino acid sequence as set forth in SEQ ID NO: 1 that exhibit enhanced tolerance to salt stress.

In an embodiment of the present disclosure, there is provided a transgenic plant or parts thereof or seeds or progeny that express a polypeptide encoded by a polynucleotide sequence as set forth in SEQ ID NO: 2 that exhibit enhanced tolerance to salt stress.

In an embodiment of the present disclosure, there is provided a transgenic plant or parts thereof or seeds or progeny that express a polypeptide having amino acid sequence as set forth in SEQ ID NO: 1 that exhibit enhanced tolerance to osmotic stress.

In an embodiment of the present disclosure, there is provided a transgenic plant or parts thereof or seeds or progeny that express a polypeptide encoded by a polynucleotide sequence as set forth in SEQ ID NO: 2 that exhibit enhanced tolerance to osmotic stress.

In an embodiment of the present disclosure, there is provided a transgenic plant or parts thereof or seeds or progeny that express a polypeptide having amino acid sequence as set forth in SEQ ID NO: 1 that exhibit enhanced tolerance to oxidative stress.

In an embodiment of the present disclosure, there is provided a transgenic plant or parts thereof or seeds or progeny that express a polypeptide encoded by a polynucleotide sequence as set forth in SEQ ID NO: 2 that exhibit enhanced tolerance to oxidative stress.

In an embodiment of the present disclosure, there is provided a transgenic plant or parts thereof or seeds or progeny that express a polypeptide having amino acid sequence as set forth in SEQ ID NO: 1 that exhibit enhanced tolerance to thermal stress.

In an embodiment of the present disclosure, there is provided a transgenic plant or parts thereof or seeds or progeny that express a polypeptide encoded by a polynucleotide sequence as set forth in SEQ ID NO: 2 that exhibit enhanced tolerance to thermal stress.

In an embodiment of the present disclosure, there is provided a transgenic plant or parts thereof or seeds or progeny that express a polypeptide having amino acid sequence as set forth in SEQ ID NO: 1 that exhibit enhanced abiotic stress tolerance.

In an embodiment of the present disclosure, there is provided a transgenic plant or parts thereof or seeds or progeny that express a polypeptide encoded by a polynucleotide sequence as set forth in SEQ ID NO: 2 that exhibit enhanced abiotic stress tolerance.

In an embodiment of the present disclosure, there is provided a transgenic plant or parts thereof or seeds or progeny that express a polypeptide having amino acid sequence as set forth in SEQ ID NO: 1 that exhibit enhanced ABA induced stress tolerance.

In an embodiment of the present disclosure, there is provided a transgenic plant or parts thereof or seeds or progeny that express a polypeptide encoded by a polynucleotide sequence as set forth in SEQ ID NO: 2 that exhibit enhanced ABA induced stress tolerance.

In an embodiment of the present disclosure, expression of OsAlba11 is maximally detected 24 hours post dehydration, when assayed at 24, 48, 72, 96, and 120 hours post dehydration.

In an embodiment of the present disclosure, transcript level of OsAlba1 is maximally detected between 48-96 hours post dehydration when assayed at 24, 48, 72, 96, and 120 hours post dehydration.

In an embodiment of the present disclosure, transcript level of OsAlba1 is maximally detected between 0-6 hours post NaCl stress, when assayed at 0, 3, 6, 12, and 24 hours.

In an embodiment of the present disclosure, transcript level of OsAlba1 is maximally detected between 3-12 hours post cold stress, when assayed at 0, 3, 6, 12, and 24 hours.

In an embodiment of the present disclosure, transcript level of OsAlba1 is maximally detected between 6-12 hours post ABA stress, when assayed at 0, 3, 6, 12, and 24 hours.

In an embodiment of the present disclosure, OsAlba1 transcript levels are maximal in flag leaf tissue.

In an embodiment of the present disclosure, OsAlba1 is localized to the nucleus.

In an embodiment of the present disclosure, OsAlba1 complements Pop6 activity in ΔPop6 yeast strain in response to oxidative stress.

In an embodiment of the present disclosure, there are provided transgenic rice plants that over-express OsAlba1, which exhibit enhanced tolerance to abiotic stress.

In an embodiment of the present disclosure, the monocot expressing a recombinant DNA construct comprising of a promoter operably linked to a gene of interest encoding a polypeptide having amino acid sequence as set forth in SEQ ID NO: 1 is selected from the group consisting of wheat, rice, banana, barley, millet, and rye.

In a preferred embodiment of the present disclosure, the monocot expressing a recombinant DNA construct comprising of a promoter operably linked to a gene of interest encoding a polypeptide having amino acid sequence as set forth in SEQ ID NO: 1 is rice.

In an embodiment of the present disclosure, the transgenic plant or part thereof including seeds and progeny expressing a recombinant DNA construct comprising of a promoter operably linked to a gene of interest encoding a polypeptide having amino acid sequence as set forth in SEQ ID NO: 1 is a dicot.

In an embodiment of the present disclosure, the dicot expressing a recombinant DNA construct comprising of a promoter operably linked to a gene of interest encoding a polypeptide having amino acid sequence as set forth in SEQ ID NO: 1 is selected from the group consisting of beans, peas, potato, eggplant, peppers, squash, melons, coffee, citrus, broccoli, turnips, yams, and apples.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

### Example 1

### Molecular Cloning of OsAlba1

The full-length cDNA of OsAlba1 was amplified from O. sativa ssp. indica (cv. Rasi) using the primers as set forth in SEQ ID NO: 3 (forward) and SEQ ID NO: 4 (reverse). The PCR mixture contained 1× reaction buffer (40 mM Tris-HCl, pH 8.0, 10 mM DTT, 60 mM KCl and 2.5% glycerol), 2.5 mM dNTPs, 2.5 U Taq DNA Polymerase, 2 mM MgCl₂, 20 pmol each of the forward and reverse primers. The reaction was conducted in a thermal cycler at the following settings: denaturation at 95° C. for 2 min, followed by 35 cycles of denaturation at 94° C. for 30 s, annealing at 65° C. for 30 s, and an extension at 72° C. for 1 min. The final extension was performed at 72° C. for 10 min. The PCR product was cloned into the pGEMT vector and confirmed by restriction analysis and sequencing.

### Example 2

### Bacterial Over-Expression of GST-OsAlba1 Fusion Protein

The OsAlba1 cDNA was inserted in-frame in to the expression plasmid pGEX4T-2 (GE Healthcare) using BamHI and NotI restriction sites engineered at 5′- and 3′-ends, respectively. The insert sequence and reading frame were verified by nucleotide sequencing, and the construct was transformed into E. coli BL21 (DE3) cells. Over-expression of recombinant protein was induced by the addition of IPTG at a final concentration of 0.3 mM. The cultures were incubated for 2 to 5 hours at 37° C. Cells were harvested by centrifugation and the recombinant protein was resolved on 12.5% SDS-PAGE and the band harboring OsAlba1 was excised from the gel, and in-gel digested with trypsin. The peptide mix generated was eluted and subjected to LC-MS/MS analysis (QSTAR Elite, AB Sciex). Multiple peptides with a high statistical probability (p<0.05) of matches to the relevant protein were analysed to confirm the identity

### Example 3

### Transcript Analysis

Total RNA was extracted from unstressed and stressed rice seedlings using TRIpure reagent (Invitrogen), and treated with RNase-free DNase I (Promega). An aliquot of 15 μg RNA was electrophoretically separated on 1.5% agarose gels and transferred onto nylon membranes (GE Healthcare) and UV cross-linked. Hybridization was performed with Alba cDNA fragment (0.459 kb) labelled with [³²P] dCTP using a NEBlot Phototype kit (New England Biolabs Inc.) under high-stringency conditions. The blot was pre-hybridized in 10 ml of hybridization buffer for at least 2 h at 60° C. with gentle rotation followed by addition of the radiolabelled probe. Following overnight hybridization, the blots were washed and exposed to pre-flashed autoradiography film, kept at −80° C. for 2 days, and developed.

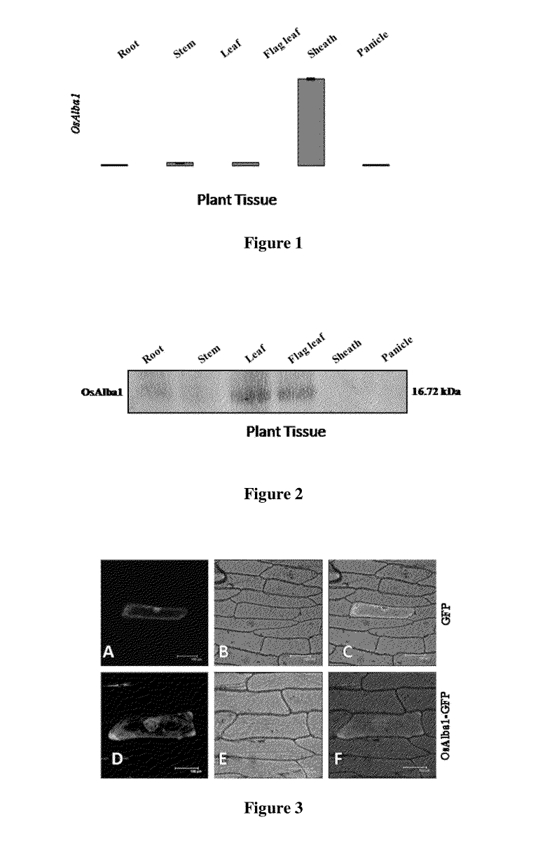

FIG. 1 shows the relative distribution of OsAlba1 gene transcript in various parts of plant. It can be inferred that maximal transcript expression of OsAlba1 can be seen in flag leaf, while there is a minimal detectable expression in other plant parts such as root, stem, leaf, and sheath. No detectable expression could be seen in panicle.

### Example 4

### Immnoblot Analysis

Immunoblot analysis was carried out by resolving nuclear proteins on a uniform 16% SDS-PAGE, and electro-transferred onto nitrocellulose membrane (GE Healthcare) at 150 mA for 3 hours. The membranes were blocked with 5% (w/v) non-fat milk for 2 hours and incubated with anti-OsAlba1 polyclonal antibody, at a dilution of 1/5000 in TBS buffer for 1 hour. The antibody was raised in rabbit against an antigenic peptide having amino acid sequence as set forth in SEQ ID NO: 5. The blot was eventually incubated with alkaline phosphatase conjugated secondary antibody, goat anti-rabbit-IgG (Sigma) at a dilution of 1/10,000 in TBS buffer for 1 hour and the signals were detected using NBT/BCIP (nitro blue tetrazolium/5-bromo-4-chloro-3-indolyl phosphate) method.

FIG. 2 shows the OsAlba11 protein distribution in various plant tissues. It can be inferred from FIG. 2 that maximal protein can be seen in flag leaf, and leaf. Background minimal expression can be detected in root, and stem of plant tissue.

### Example 5

### Sub-Cellular Localization of OsAlba1

Inner epidermal peels of young onion bulb were placed on MS-agar plates supplemented with 3% sucrose. Approximately 25 μg of OsAlba1 construct plasmid was coated onto 1.6 μm gold particle (Bio-Rad) with 2.5 M CaCl₂ and 0.1 M spermidine, and mixed vigorously. Plasmid-coated particles were dehydrated with 75% and 95% ethanol prior to bombardment and introduced transiently into epidermal cells by microprojectile bombardment using a PDS-1000/He-driven particle accelerator (Bio-Rad). The samples were incubated for 22-24 h under dark conditions at 22° C. before analysis with TCS SP2 confocal system (Leica, Germany) for fluorescence detection. GFP signals were detected between 505-550 nm after exciting with 488 nm. Co-localization was performed with sequential scanning for both the channels and then merged together to obtain overlapping signals.

FIG. 3 shows the sub-cellular localization of OsAlba1-GFP fusion protein. It can be seen that GFP expression shows accumulation in nucleus (FIG. 3D), and sparsely in the cytoplasm, compared to control GFP alone (FIG. 3A). FIGS. 3B, and 3E are phase contrast images of same cells as FIG. 3A, D. FIG. 3C, F are merged images of FIGS. 3A, B and D, E respectively.

To determine the organ-specific expression of OsAlba1, transcript abundance in roots, stems, leaves, flag leaves, sheaths and panicles of rice seedlings were compared. The transcripts were abundantly and constantly transcribed in flag leaves. The expression in other major vegetative organs was substantially low (FIG. 1). Next, the protein abundance in all these tissues was examined by immunoblot analysis (FIG. 2). The results showed the presence of OsAlba1 in leaves and flag leaves, corroborating the mRNA profile. The predominant expression of OsAlba1 in the flag leaves seems to support the growing panicles which might be crucial during grain filling. The rice flag leaves are essential in providing photosynthates (Xu et al., PloS One, 2011, 6, e176133) and has been reported to play an important role in grain yield (Wan et al., Scienctia Agricultura Sinica, 1981, 6, 21-28; Raj et al., Indian J. Plant Physiol., 2000, 5, 293-295) and in enhancing productivity (Padmaja, Indian J. Plant Physiol., 1991, 34, 339-348). These results may have important implications, since such organ-specific gene expression is closely related to harvest yield, and perhaps crucial for driving agronomic trait/s.

### Example 6

### Complementation Study in Yeast

S. cerevisiae wild-type (BY4743) and ΔPop6 (YGR030c) strains were used for complementation assay. ΔPop6 was transformed with pYES2 vector and pYES2 vector containing the OsAlba1 ORF. Yeast strains were grown in YPD broth and serial dilutions [OD₆₀₀=0.5, as 10° to 10⁻³ for ΔPop6] were prepared and plated. The plates were incubated at 30° C. for 48 h. The growth of wild-type (WT), ΔPop6:pYES2, ΔPop6, and ΔPop6+OsAlba1 strains were checked under normal conditions, as well as under H₂O₂-induced oxidative stress conditions.

Ribonuclease P/MRP subunit Pop6, a component of ribonuclease P, is an Alba domain containing protein involved in RNA processing (Mani et al., PLoS One., 2011, 6, e22463). However, the role of Alba super family proteins in stress tolerance remains unexplored. It is well known that oxidative stress is caused by increased production of reactive oxygen species (ROS) that damage various cellular components, including proteins, lipids and DNA. We thought that OsAlba1, being a DNA-binding protein, might be involved in defense against oxidative damage and therefore, its role in oxidative stress tolerance was investigated. We examined whether OsAlba1 could complement yeast ΔPop6 mutant under oxidative stress.

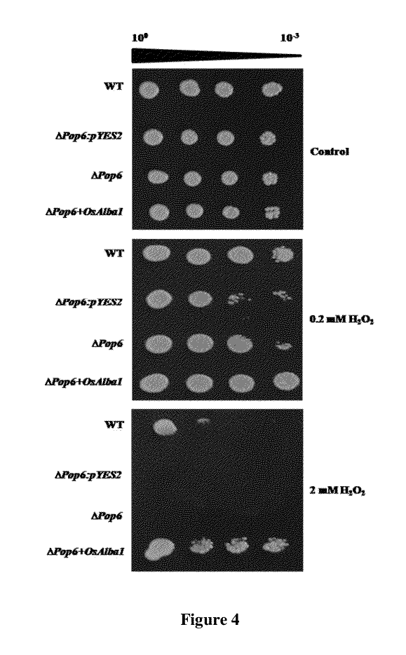

FIG. 4 shows that the growth of wild-type (WT), ΔPop6:pYES2, ΔPop6 and complemented (ΔPop6+OsAlba1) strains was monitored in presence of different concentrations of H₂O₂. The complemented strains grew rapidly and displayed resistance when compared with that of ΔPop6 and ΔPop6:pYES2, and the effect was more prominent at higher concentration of H₂O₂. These results indicate the role of Alba proteins in oxidative stress tolerance, besides their classical role as DNA/RNA-binding proteins.

### Example 7

### Plant Growth Conditions and Stress Treatments

Rice (Oryza sativa L. ssp. indica) seedlings were grown in pots containing a mixture of soil and soilrite (2:1, w/w; 10 plants/5.6-l-capacity pot) in an environmentally controlled growth room. The seedlings were maintained at 28±2° C., 70±5% relative humidity under a 16 h photoperiod (270 μmol/m²/second light intensity). The pots were provided with 300 ml of water every day to maintain the soil moisture content at ˜30%. Dehydration stress was imposed on the 4-week-old seedlings by withdrawing water, and tissues were harvested up to 120 h.

In a separate set of experiment, the seedlings were supplemented with half-Hoagland's medium until 4 weeks, followed by treatment with 250 mM NaCl in the medium. The tissues were harvested until 24 h of the treatment. The ABA treatment was carried out by spraying the seedlings with 100 μM ABA, and tissues were harvested at specified intervals. The unstressed seedlings were sprayed with water, referred to as 0 h. For cold treatment, seedlings were kept at 4° C. for a period of 24 h.

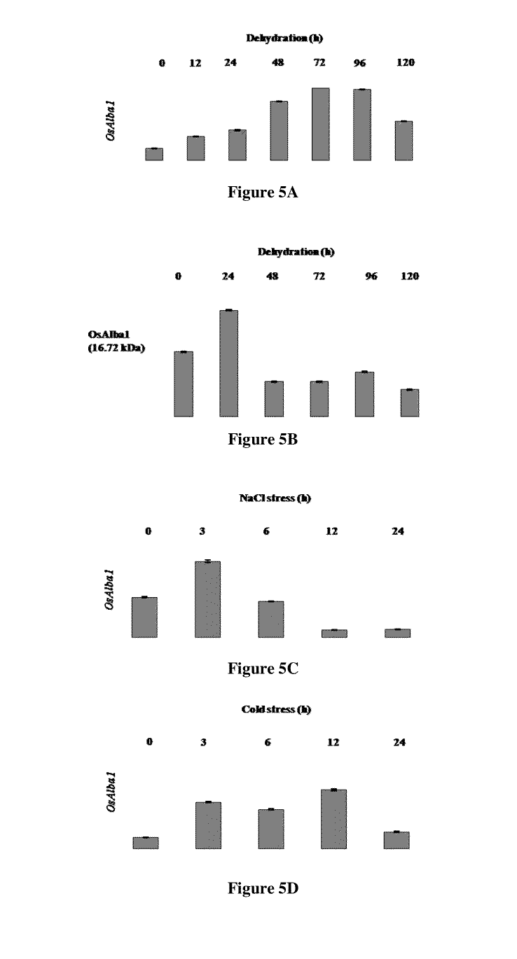

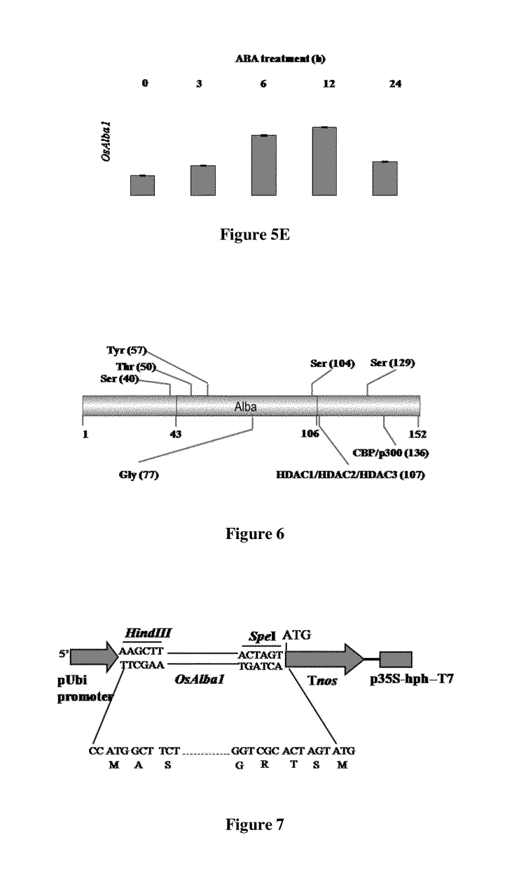

In order to investigate transcriptional regulation of OsAlba1 under dehydration, we carried out Northern blot analysis (FIG. 5). The OsAlba1 transcripts were induced during 48-96 h, but dropped to steady state level at 120 h of dehydration (FIG. 5A). The discrepancy between the transcript abundance and the protein profile (FIG. 5B) is not unprecedented and suggests posttranscriptional and/or posttranslational regulation of OsAlba1 during stress. Because dehydration-responsive pathway often overlaps with that of ABA, and dehydration represents a common stress challenge to plant cells under high salinity and cold conditions (Thomashow, Annu. Rev. Plant Physiol. Plant Mol. Biol. 1999, 50, 571-599, Shinozaki et al., Curr. Opin. Plant Biol. 3, 2000, 217-223; Xiong et al., Plant Cell Environ., 2002, 25, 131-139), the expression of OsAlba1 transcripts was investigated triggered by each of these conditions. A persistent induction of OsAlba1 was observed under cold, with maximum transcript accumulation at 12 h of stimulation. Even at 24 h of cold treatment, the mRNA level remained elevated when compared with the unstressed condition (FIG. 5D). Treatment with 250 mM NaCl resulted in somewhat more transient mRNA elevation that declined within 24 h (FIG. 5C). Next, we examined whether OsAlba1 transcription was affected by ABA, and it appeared to be induced until 12 h of treatment (FIG. 5E). These results altogether suggest that OsAlba1 may actively participate in osmotic and hyper-osmotic stress, and this participation may, in part, depend on ABA.

### Example 8

### Structural Organization of OsAlba1

To investigate the genomic organization of OsAlba1, the NCBI sequence database was reviewed. It revealed the presence of an incomplete transcript with a coding region of 0.407 kb, identical to Alba protein from Oryza sativa ssp. japonica. The corresponding sequence was used to design primers and full-length cDNA was cloned from O. sativa ssp. indica. The analysis revealed a 52 bp sequence missing at 3′-end, identical to O. sativa ssp. japonica. The complete OsAlba1 genomic sequence is thus 1.514 kb, excluding 3′-UTR region, and interrupted by 4 introns. The coding region of OsAlba1 encodes a protein of 152 amino acids with molecular mass of 16.72 kDa.

The domain architecture and putative post translational modifications were examined using NCBI CDD server for domain prediction and NetPhos 2.0 for predicting the phosphorylation sites. The deduced protein sequence analysis and domain search indicated that OsAlba1 comprises of 64 amino acid residue Alba domain (Pfam Accession: PF01918). OsAlba1 showed a high score phosphorylation, both within the Alba domain (Thr-50, Tyr-57 and Ser-104) and outside it (Ser-40 and Ser-129) (FIG. 6). Phosphorylation has previously been observed in Alba proteins of Pop7 and RPP25 family from mouse and human and is documented in UniProt database. Using ASEB server, acetylation and deacetylation sites (acetylation, 136; deacetylation, 107) in OsAlba1 were also predicted. Acetylation and deacetylation events have earlier been reported to have important roles in differential regulation (Chene et al., Nucleic Acids Res., 2012, 40, 3066-3077) and a similar mechanism of differential regulation of OsAlba1 by PTM could therefore be suggested.

### Example 9

### Rice Transformation

### Agrobacterium Strains and Construct Used for Transformation

The OsAlba1 gene was cloned in pGA3426 (FIG. 7) plant transformation vector at XbaI-KpnI restriction sites between the maize Ubi promoter and nos terminator. This vector has tet (tetracycline) and hph (hygromycin) genes as the selectable markers for bacteria and plants, respectively. The gene construct was finally transformed in Agrobacterium tumefaciens, GV3101.

Primary culture of Agrobacterium was prepared by inoculating a single colony from a freshly streaked plate, in 5 ml of YEP medium (10 g/l bactopeptone, 10 g/l yeast extract, 5 g/l sodium chloride, pH adjusted 7.0) supplemented with 10 mg/l tetracycline and 10 mg/l rifampicin. The culture was incubated at 28° C. for 16-20 h on a rotatory incubator shaker at 200 rpm. Secondary culture was prepared in a 500 ml baffled flask containing 100 ml YEP by adding 1.0% of the primary culture and grown under similar conditions. Once the O.D.₆₀₀ reached ˜1.0, Agrobacterium cells were pelleted by centrifugation at 8000×g for 15 min at 4° C. The cells were resuspended in MS resuspension medium containing 150 μM acetosyringone to adjust the O.D.₆₀₀ of the bacterial suspension to 0.3.

### Agrobacterium Mediated Callus Transformation and Regeneration

The 2-week-old rice calli were collected and Agro-infected by immersing them in the Agrobacterium culture for 20-25 min with intermittent gentle shaking. The Agro-infected calli were dried on sterile Whatman filter paper for 5 min. Calli were then transferred to the co-cultivation medium containing 30 g/L sucrose, 150 μM acetosyringone and incubated at 27±1° C. in the dark for 3 d. Once slight growth of Agrobacterium appeared around most of the calli, the calli were rinsed 8-10 times with 250 mg/L cefotaxime. They were then dried on sterile Whatman filter paper and transferred onto first selection shooting medium (MS medium, BAP 3 mg/l, NAA 1 mg/l, 250 mg/l cefotaxime and 50 mg/l hygromycin and incubated for 12 d at 26±1° C. in dark. After the first selection, brown or black calli were removed, and only creamish healthy calli were transferred to the second selection and maintained at 26±1° C. in dark. After second selection for 10 d, microcalli could be observed which were finally transferred to fresh MS shooting media for third selection and allowed to proliferate for 5 d at 27±1° C. in dark.

Healthy shoots with defined stem were transferred to rooting medium (MS medium supplemented with IBA 1 mg/l, 50 mg/l hygromycin, 500 mg/l cefotaxime, pH 5.6) and incubated at 28° C. under continuous light. The plantlets with well-developed root system were planted in 250 ml plastic pots containing autoclaved mud, collected from rice fields. The plantlets were established in such a way that the clump of plantlets, originated from each callus, was separated carefully and planted in several pots. Out of different transgenic lines obtained, five independent transgenic lines harbouring OsAlba1 were screened for further analyses.

### Example 10

### Analysis of OsAlba1 Over-Expressing Transgenic Plants for Stress Tolerance

The wild type and the transgenic plants are being studied in parallel in the same growth room for comparative morpho-anatomical, molecular, and physiological analyses. The rice seedlings have been grown in pots containing a mixture of soil and soilrite (2:1, w/w; 5.6-liter-capacity pot) in an environmentally controlled growth room. The seedlings are maintained at 28±2° C., 70±5% relative humidity under a 16 h photoperiod (270 μmol m⁻² s⁻¹ light intensity). The pots are provided with 300 ml of water every day that maintained the soil moisture content at 30%. Dehydration is imposed on the 4-week-old seedlings by withdrawing water, and tissues will be harvested up to 120 h. The harvested tissues will be instantly frozen in liquid nitrogen and stored at −80° C. unless otherwise described. In a separate set of experiment, the seedlings have been supplemented with half-Hoagland's medium for 3 weeks, followed by treatment with 250 mM concentration of NaCl in the same medium. The tissues will be harvested 24 h after the treatment. The treatment of ABA will be carried out by spraying the seedlings with 100 μM ABA, and tissues will be harvested at specified intervals. For cold treatment, seedlings have been kept at 4° C. for 24 h. The transcript analysis of OsAlba1 for its differential expression under various stresses will be carried out by Northern blot and quantitative real-time PCR.

Overall, the present disclosure provides transgenic plants, methods and reagents to generate transgenic plants that surprisingly exhibit enhanced tolerance to a variety of abiotic stress factors.

What is claimed is:

1. A recombinant DNA construct comprising of a promoter operably linked to a gene of interest, wherein said gene of interest encodes a polypeptide having amino acid sequence as set forth in SEQ ID NO: 1.

2. The recombinant DNA construct as claimed in claim 1, wherein the gene of interest polynucleotide sequence is as set forth in SEQ ID NO: 2.

3. The recombinant DNA construct as claimed in claim 1 or 2, wherein the promoter is selected from the group consisting of pUbi, CamV 35S, Act-1, Adh-1, and opine promoters.

4. A recombinant DNA vector comprising the recombinant DNA construct as claimed in claim 1 or 2.

5. A recombinant host cell comprising the recombinant DNA construct as claimed in claim 1 or 2 or the recombinant DNA vector as claimed in claim 4.

6. The recombinant host cell as claimed in claim 5, wherein said recombinant host cell is of bacterial or fungal or of plant origin.

7. A cDNA fragment encoding a polypeptide having amino acid sequence as set forth in SEQ ID NO: 1, wherein recombinant cells expressing said polypeptide exhibit enhanced abiotic stress tolerance selected from the group consisting of ABA induced stress, salt stress, thermal stress, dehydration stress, oxidative stress, and combinations thereof.

8. The cDNA as claimed in claim 7, wherein the cDNA polynucleotide sequence is as set forth in SEQ ID NO: 2.

9. A method of producing a transgenic plant with enhanced tolerance to abiotic stress, said method comprising:
a. transforming plant cells with a recombinant DNA construct as claimed in claim 1 or recombinant host cells as claimed in claim 5;
b. selecting a transgenic cell; and
c. developing a transgenic plant from said transformed cell,
wherein said transgenic plant exhibits enhanced tolerance to abiotic stress.

10. The method as claimed in claim 9, wherein said transgenic plant is a monocot or a dicot.

11. The method as claimed in claim 9, wherein the abiotic stressors are selected from the group consisting of ABA induced stress, salt stress, thermal stress, dehydration stress, oxidative stress, and combinations thereof.

12. A transgenic plant or parts thereof, including seeds, wherein said transgenic plant or parts thereof, including seeds, encodes at the nuclear genome level a polynucleotide fragment comprising of a cDNA fragment encoding a polypeptide having amino acid sequence as set forth in SEQ ID NO: 1, wherein expression of said polypeptide confers abiotic stress resistance to said transgenic plant or parts thereof, including seeds.

13. The transgenic plant or parts thereof, including seeds as claimed in claim 12, wherein said cDNA has a polynucleotide sequence a set forth in SEQ ID NO: 2.

14. The transgenic plant or parts thereof, including seeds as claimed in claim 12, wherein said transgenic plant or parts thereof, including seeds is a monocot or a dicot.

15. Use of a cDNA fragment encoding a polypeptide having amino acid sequence as set forth in SEQ ID NO: 1 in generating transgenic plants which are resistant to abiotic stress.


SEQUENCE LISTING (5 sequences)

SEQ ID NO: 1 - protein, 152 aa, artificial sequence
  OsAlba1 protein
MAVEEITEGVRNLAVEGEPAAAAAAAGGGGEGAQRRAAGSSSNRIQVSNTKKPLFFYVNLAKRYMQQHGDVELSALGMAIATVVTVAEILKNNGFAVEKKIRTSTVEINDESRVRPLQKAKIEIVLEKSEKFDELMAAAAEEREAAEAEEQA

SEQ ID NO: 2 - DNA, 459 bp, artificial sequence
  OsAlba1 cDNA
atggcggtggaggagatcaccgagggggtgaggaacctggccgtggagggggagcccgcggcggcggcggcggcggcgggaggtggtggtgagggggcgcagaggagggcggccgggagcagcagcaaccgcatccaggtgtccaacaccaagaagccactcttcttctatgtcaacctcgccaagaggtacatgcagcagcacggcgatgtcgagctctccgcgctcgggatggccattgcaacagttgtaactgttgcggagattcttaagaataacgggtttgctgttgaaaagaagattagaacatctacggtggaaataaacgatgaatcgagagttcgcccgctccaaaaggctaagattgagatagtgttagaaaagagcgagaaatttgatgagctgatggctgccgcagcggaagagagggaagctgcggaagctgaggagcaggcctga

SEQ ID NO: 3 - DNA, 28 bp, artificial sequence
  OsAlba1 forward primer
cgcggatccatggcggtggaggagatca

SEQ ID NO: 4 - DNA, 32 bp, artificial sequence
  OsAlba1 reverse primer
ataagaatgcggccgctcaggcctgctcctca

SEQ ID NO: 5 - protein, 15 aa, artificial sequence
  OsAlba1 peptide ab antigen
RTSTVEINDESRVRP